(19) United States
(12) Patent Application Publication
(10) Pub. No.: US 2024/0082069 A1
(43) Pub. Date: Mar. 14, 2024

(54) A METHOD FOR LOCATING A VACUUM ASSISTED THERAPY DEVICE AND A DEVICE FOR USE IN THE METHOD

(71) Applicant: Daniel Eduard KLEINER, Makara (NZ)

(72) Inventor: Daniel Eduard KLEINER, Makara (NZ)

(21) Appl. No.: 18/274,780

(22) PCT Filed: Jan. 25, 2022
(86) PCT No.: PCT/AU2022/050033

§ 371 Date: Jul. 28, 2023

Related U.S. Application Data
(60) Provisional application No. 63/141,212, filed on Jan. 25, 2021.

(30) Foreign Application Priority Data
Oct. 1, 2021 (AU) 2021903161

(51) Int. Cl. A61F 13/00; A61F 13/02; A61M 1/00; A61M 39/08

(52) U.S. Cl. CPC A61F 13/00068; A61F 13/0216; A61M 1/96; A61M 39/08; A61M 2039/082

(57) ABSTRACT

There is described a vacuum assisted therapy device (36) for applying a negative pressure to a site in an endoluminal surface in the upper gastrointestinal tract 5 of a patient. In embodiments, the device (36) comprises an absorption element (38) for application of suction to the endoluminal surface via an outer peripheral face (71) of the absorption element. The outer peripheral surface extends between the proximal and distal ends of the absorption element. The device also comprises tubing (40) providing a longitudinal internal lumen for supplying the suction to the absorption element (38), 10 and a coupling fitting (50) is provided on a distal end of the tubing. The coupling fitting is configured for connection in use to an extraction element (60) for drawing of the distal end of the tubing through the nasal cavity of the patient following insertion of the absorption element into the upper gastrointestinal tract of the patient via the patient's mouth. The tubing (40) can provide one or more internal lumens to the 15 absorption element. In embodiments, a coupling (50, 58) formed between the extraction element and the coupling fitting of the device is sized and dimensioned to facilitate the drawing of the tubing through the nose of the patient. Methods for use of the use of the vacuum assisted therapy device (36) are also described. 20

### FIELD OF THE INVENTION

The present invention relates to the field of vacuum assisted therapy for the treatment of endoluminal surfaces of the upper respiratory tract of a patient.

### BACKGROUND OF THE INVENTION

The application of sub-atmospheric pressure to acute or chronic wounds to promote wound healing is known as negative pressure wound therapy (NPWT) or vacuum assisted closure (VAC). VAC therapy involves creating a negative-pressure in the local wound environment, drawing away bacteria, exudate, fluid and desiccated tissue from the wound site. Besides improving localised conditions and reducing oedema for wound healing, the negative pressure may draw wound edges together and increase the rate of healing by promoting blood flow and facilitating localised cell migration and proliferation. Indeed, it is believed VAC therapy can increase the rate of wound closure.

Conventionally, VAC therapy has been applied to wounds in the skin such as burns, grafts, surgical incisions, diabetic ulcers, pressure ulcers, venous stasis ulcers and wounds arising from trauma. These “wound VAC” devices comprise a pad of open-cell sponge like material or a porous mat for being placed on the wound. A vacuum is applied to the sponge via a drainage tube through which fluid and exudate from the wound that is drawn into the sponge or mat is drained away. A drape can be laid over the sponge or porous mat to facilitate sealing of the wound. Such devices are commercially available and, for example, are described in U.S. patent application Ser. No. 11/186,056, U.S. Ser. No. 11/347,073, U.S. Ser. No. 11/409,116, U.S. Ser. No. 11/268,212, U.S. Ser. No. 12/233,211, and International Patent Application WO 93/09727. In more recent times, VAC devices comprising like porous sponges and mats have been used to drain seromas and fluids from internal bodily spaces following surgery and to facilitate the healing of wounds on outer surfaces of internal body organs and tissues, examples of which are described in WO 03/028786, U.S. Pat. No. 5,437,651 and patent application U.S. Ser. No. 11/646,918.

Further vacuum assisted therapy devices are described in WO 2012/071626 and WO 2013/181686, both by the present inventor.

### SUMMARY OF THE INVENTION

Broadly, the present invention in one or more forms relates to a method for locating a vacuum assisted therapy device in location in the upper gastrointestinal tract of a patient and device suitable for use in methods as described herein.

More particularly, in one aspect of the present invention there is provided a method for locating a vacuum assisted therapy device in the upper gastrointestinal tract of a patient, comprising:
providing a vacuum assisted therapy device for application of negative pressure to a site in an endoluminal surface of the upper gastrointestinal tract, the device having an absorption element with an outer peripheral face for contact with the endoluminal surface, and tubing providing a longitudinal internal lumen for being connected to a suction source, the lumen of the tubing being in fluid communication with the absorption element to apply the negative pressure to the endoluminal surface via the outer peripheral face of the absorption element with operation of the suction source;
inserting the absorption element in the upper gastrointestinal tract via the mouth of the patient whereby the tubing of the device extends from the patient's mouth;
inserting an extraction element though the nose of the patient into the throat of the patient, the extraction element having a proximal end and an opposite distal end;
withdrawing the proximal end of the extraction element from the mouth of the patient leaving the distal end of the extraction element exterior of the patient's nose;
connecting the proximal end of the extraction element to a distal end of the tubing of the device; and
withdrawing the extraction element from the nose of the patient whereby the distal end of the tubing of the device is drawn back into the throat of the patient and out of the patient's nose whereby the absorption element remains within the upper gastrointestinal tract of the patient.

Typically, the withdrawal of the extraction element from the mouth of the patient comprises inserting an elongate retrieval element into the patient's mouth, using the retrieval element to grip or couple to the extraction element, and withdrawing the retrieval element to withdraw the proximal end of the extraction element from the mouth of the patient.

Most typically, the retrieval element magnetically couples to the extraction element for withdrawal of the proximal end of the extraction element from the mouth of the patient.

Typically, the retrieval element includes a permanent magnet and the extraction element includes a magnetically attracted material, or vice versa, and the permanent magnet magnetically couples to the magnetically attracted material. In particularly preferred embodiments, the retrieval element includes the permanent magnet and the extraction element is provided with the magnetically attracted material.

In at least some embodiments, the permanent magnet or the magnetically attracted material as applicable is removable from the extraction element.

Typically, the extraction element is provided with a removeable end cap for magnetically coupling with the permanent magnet or the magnetically attracted material of the retrieval element.

Typically, the retrieval element is a probe wherein the permanent magnet or the magnetically attracted material as applicable is mounted to an end of the probe.

Typically, the distal end of the tubing of the device is provided with a coupling fitting for coupling to the proximal end of the extraction element for drawing of the distal end of the tubing of the device from the nose of the patient with the withdrawal of the extraction element.

Most typically, a method embodied by the invention further comprises either:
(i) disconnecting the extraction element from the tubing of the device after withdrawing the extraction element from the nose of the patient, and connecting further tubing to the distal end of the tubing of the device, the further tubing having a coupling fitting on the proximal end of that tubing, and mating the coupling fitting of the further tubing with a coupling fitting of the tubing of the device to form a coupling connecting the tubing of the device to the further tubing whereby the lumen of the tubing of the device is in fluid communication with a corresponding longitudinal internal lumen of the further tubing; or
(ii) wherein the extraction element comprises further tubing provided with a coupling fitting on the proximal end of that tubing for mating with a coupling fitting provided on the distal end of the tubing of the device, and the connecting of the extraction element to the tubing of the device comprises mating the coupling fitting of the further tubing with the coupling fitting on the tubing of the device to form a coupling connecting the tubing of the device to the further tubing so the lumen of the tubing of the device is in fluid communication with a corresponding longitudinal internal lumen of the further tubing;
whereby the distal end of the tubing of the device is drawn from the patient's nose with said withdrawal of the extraction element.

In another aspect of the invention there is provided a vacuum assisted therapy device for applying a negative pressure to a site in an endoluminal surface in the upper gastrointestinal tract of a patient, the device comprising:
an absorption element for application of suction to the endoluminal surface via an outer peripheral face of the absorption element, the absorption element having a proximal end and an opposite distal end, and the outer peripheral surface extending between the proximal and distal ends of the absorption element;
tubing providing a longitudinal internal lumen for supplying the suction to the absorption element; and
a coupling fitting provided on a distal end of the tubing and configured for connection of the tubing in use to an extraction element for drawing of the distal end of the tubing through the nasal cavity of the patient to protrude from the nose of the patient following insertion of the absorption element into the upper gastrointestinal tract of the patient via the patient's mouth.

From the above, in embodiments described herein, the coupling fitting of the tubing and thereby the coupling formed by the coupling fitting and the extraction element, is adapted (e.g., shaped and dimensioned) for being drawn through the nasal passage and from the nose of the patient.

Typically, the lumen of the tubing of the device for supplying the suction to the absorption element is one of a plurality of longitudinal internal lumens of that said tubing which lead to the absorption element.

Likewise, the internal lumen of the further tubing is typically one of a plurality of longitudinal internal lumens of the further tubing, and wherein each said lumen of the tubing of the device is aligned with a corresponding one of the lumens of the further tubing when the coupling fittings of the tubing of the device and the further tubing are mated together whereby each said lumen of the tubing of the device is in fluid communication with the corresponding said lumen of the further tubing.

In particular, in another aspect of the invention there is provided a vacuum assisted therapy device for applying a negative pressure to a site in an endoluminal surface in the upper gastrointestinal tract of a patient, the device comprising:
an absorption element with an outer peripheral face for contact with the site in the endoluminal surface; and
tubing to which the absorption element is connected, the tubing providing a plurality of longitudinal internal lumens, the longitudinal lumens including a suction lumen for being connected to a suction source external of the body of the patient and being in fluid communication with the absorption element to apply the negative pressure to the endoluminal surface via the outer peripheral face of the absorption element with operation of the suction source; and
a coupling fitting for being connected to further tubing comprising a plurality of further longitudinal internal lumens whereby respective of the lumens of the tubing of the device are aligned with corresponding ones of the lumens of the further tubing to form respective conduits to the absorption element, the further tubing being for location external of the patient, and wherein the coupling fitting is provided on an opposite end of the tubing of the device to the absorption element.

Typically, the absorption element has at least one through passageway extending from its proximal end and opening to its distal end and wherein the proximal and distal ends of the absorption element are otherwise sealed against egress of bodily fluids present within the upper gastrointestinal tract of the patient into the absorption element.

In at least some embodiments, the device further comprises at least one absorbent component for absorbing bodily fluids present within the upper gastrointestinal tract of the patient, the absorbent component being respectively located proximally or distally to the absorption element and separated from the absorption element by a juncture connecting the absorbent component to the absorption element, the juncture sealing the absorption element against egress of said bodily fluids from the absorbent component into the absorption element.

Typically, the further tubing of a device embodied by the invention comprises the extraction element for withdrawing the distal end of the tubing of the device from the nose of the patient in accordance with a method embodied by the invention.

Typically, the coupling fitting of the device is provided for mating with the coupling fitting of the further tubing to form a respective sealed connection between each said lumen of the tubing of the device and the corresponding said lumen of the further tubing.

Typically, the tubing of the device comprises an outer tube and inner tubing received within the outer tube, the lumens of the device being defined by the inner tubing.

Typically, the inner tubing comprises a plurality of tubes each defining a respective longitudinal said lumen of the tubing of the device.

Typically, the lumens of the device comprise one or more lumens opening to beyond the proximal end of the absorption element thereby providing passage through the absorption element to forward of the absorption element.

Typically, each said further lumen opening to beyond the proximal end of the absorption element is respectively a lumen independently selected from the group consisting of a drainage lumen for drainage from the gastrointestinal tract to exterior of the patient, an access lumen for providing access to the gastrointestinal tract, and a lumen for passage of food and/or administered liquids from exterior of the patient through the absorption element into the gastrointestinal tract of the patient.

Typically, at least one further said lumen of the device opens into the absorption element in addition to the suction lumen. In at least some embodiments, a plurality of said lumens of the device open into the absorption element in addition to the suction lumen. Each said lumen opening into the absorption element in addition to the suction lumen can independently be selected from the group consisting of a lumen for providing irrigation fluid internally into the absorption element, a further suction lumen for providing the suction to the peripheral face of the absorption element, and a lumen for passage of a fluid into the absorption element for expanding the absorption element to press the outer peripheral face of the absorption element against the endoluminal surface. The fluid for expanding the absorption element to press against the endoluminal surface may be air or other suitable gas, or a liquid.

Typically, the mating of the coupling fittings of the tubing of the device and the further tubing comprises securing the coupling fittings together.

In yet another embodiment, a vacuum assisted therapy device of the invention may further comprise a tag for being gripped to draw the device along the upper gastrointestinal tract of the patient to locate the outer peripheral surface of the absorption element adjacent the site in the endoluminal surface to be treated.

Hence, in yet another aspect of the invention there is provided a vacuum assisted therapy device for applying a negative pressure to a site in an endoluminal surface in the upper gastrointestinal tract of a patient, the device comprising:
an absorption element for application of suction to the endoluminal surface via an outer peripheral face of the absorption element for contact with the endoluminal surface, the absorption element having a proximal end and an opposite distal end, and the outer peripheral surface extending between the proximal and distal ends of the absorption element;
tubing for supplying the suction to the absorption element; and
a tag for being gripped to draw the device along the upper gastrointestinal tract of the patient to locate the outer peripheral surface of the absorption element adjacent the site in the endoluminal surface to be treated, the tag being located forward of the proximal end of the absorption element.

In embodiments in accordance with this aspect of the invention, the tag can be affixed to any suitable support member of the device located forwardly of the proximal end of the absorption element.

Typically, an inner tube (e.g., a drainage inner tube) of the tubing of the device protrudes from the proximal end of the absorption element and the tag is affixed to the protruding region of the inner tube.

Still further, in another aspect of there is provided a method for locating a vacuum assisted therapy device within the upper gastrointestinal tract of a patient for applying a negative pressure to a site in an endoluminal surface in the upper gastrointestinal tract, the method comprising:
providing the vacuum assisted therapy device, the device having an absorption element with an outer peripheral face for application of applied suction to the endoluminal surface, the absorption element having a proximal end and an opposite distal end, and the outer peripheral surface extending between the proximal and distal ends of the absorption element, and the device further comprising tubing for supplying the suction to the absorption element, and a tag for being gripped to draw the device along the upper gastrointestinal tract of the patient into position whereby the outer peripheral surface of the absorption element is located alongside the site in the endoluminal surface to be treated, the tag being provided forward of the proximal end of the absorption element;
inserting the absorption element into the upper gastrointestinal tract of the patient; and
drawing the absorption element along the gastrointestinal tract of the patient via the tag to locate the outer peripheral face of the absorption element alongside the site in the endoluminal surface.

In still another aspect of the invention there is provided a kit for locating a vacuum assisted therapy device within the upper gastrointestinal tract of a patient for applying a negative pressure to a site in an endoluminal surface in the upper gastrointestinal tract, the kit comprising a vacuum assisted therapy device as described herein in combination with the extraction element for being coupled to the distal end of the tubing of the device to facilitate withdrawal of the distal end of the tubing from the nose of the patient in accordance with the present disclosure. The kit can also comprise the retrieval element for retrieving the proximal end of the extraction element from the mouth of the patient.

Advantageously, a vacuum assisted therapy device located in position in the upper respiratory tract in accordance with a method as described herein can be removed by pushing or withdrawing the distal end of the tubing of device back through the patient's nose and then withdrawing the distal end of the tubing from the patient's mouth to thereby allow for the absorption element of the device to be removed from their mouth by pulling on the tubing of the device.

The location and removal of the vacuum assisted therapy device via the mouth of the patient thereby avoids discomfort and trauma to the patient that may otherwise be associated with location and/or removal of the absorption element via the patient's nasal cavity. Further, as the tubing of the device when located in position in the upper gastrointestinal tract exits via the nose of the patient, providing for patient comfort and allowing for endoscopic investigation or introduction via the mouth of the patient.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or elsewhere before the priority date of this application.

The features and advantages of the present invention will become further apparent from the following detailed description of exemplary embodiments of the invention together with the accompanying drawings.

### BRIEF DESCRIPTION OF THE ACCOMPANYING DRAWINGS

FIG. 1 is a diagrammatic perspective view of a prior art vacuum assisted therapy device;

FIG. 2 is a diagrammatic side view of absorption elements of another prior art vacuum assisted therapy device;

FIG. 3 is a diagrammatic side view of a vacuum assisted therapy device embodied by the present invention;

FIG. 3a is a diagrammatic side sectional view of a tubing end cap component of a coupling fitting of the device of FIG. 3;

FIG. 3b is an end view of a tubing end cap component;

FIG. 3c is a diagrammatic side sectional view of a further tubing end cap component of an embodiment of the invention;

FIG. 4 is a diagrammatic view of the upper gastrointestinal (GI) tract of a patient;

FIG. 5 is a diagrammatic view illustrating the insertion of the absorption element of a vacuum assisted therapy device embodied by the invention into position in the upper GI tract;

FIG. 6 is a diagrammatic view illustrating the insertion of an extraction element in the form of further tubing through the nose of the patient into position at the back of the patient's throat;

FIG. 7 is a diagrammatic view of illustrating the coupling of a retrieval element with the extraction element of FIG. 6;

FIG. 8 is a diagrammatic view illustrating the retrieval of the distal end of the tubing of the vacuum assisted therapy device of claim 5 from the patient's mouth;

FIG. 9 is a diagrammatic view illustrating coupling of the tubing of the vacuum assisted therapy device of claim 8 with the extraction element;

FIG. 10 is a diagrammatic view illustrating the distal end of the tubing of the vacuum assisted therapy device of claim 9 after being drawn through the nasal cavity of the patient with withdrawal of the extraction element; and

FIG. 11 is a diagrammatic view of another vacuum assisted therapy device embodied by the invention.

### DETAILED DESCRIPTION OF EXEMPLARY EMBODIMENTS OF THE INVENTION

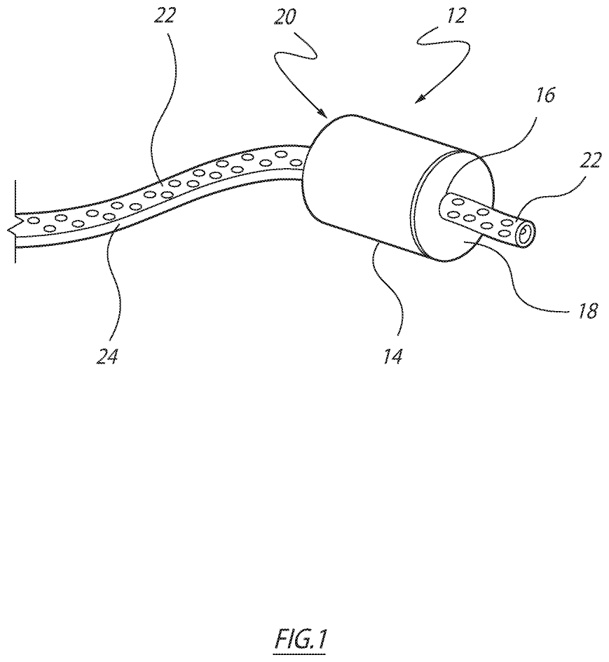

The prior art vacuum assisted therapy device shown in FIG. 1 comprises an absorption element 12 in the form of a biocompatible foamed sponge with an outer peripheral face 14 for contact with an endoluminal surface of a patient. A central through passageway 16 extends from a proximal end 18 of the sponge to an opposite distal end 20. A drainage tube 22 is received in the through passageway of the sponge and allows for passage of bodily substances e.g., faecal matter, gases etc through the sponge from the lumen in which the sponge 12 is placed for drainage to exterior of the patient. A separate suction tube 24 for connection to a suction source located externally of the patient's body is in fluid communication with the sponge 12 for application of a negative pressure to the sponge with operation of the suction source. Both the proximal and distal ends of the sponge are essentially impermeable to gases and fluids, and so are sealed against egress of the bodily substances into the sponge under the action of the suction applied to the sponge in use via the suction tube 24.

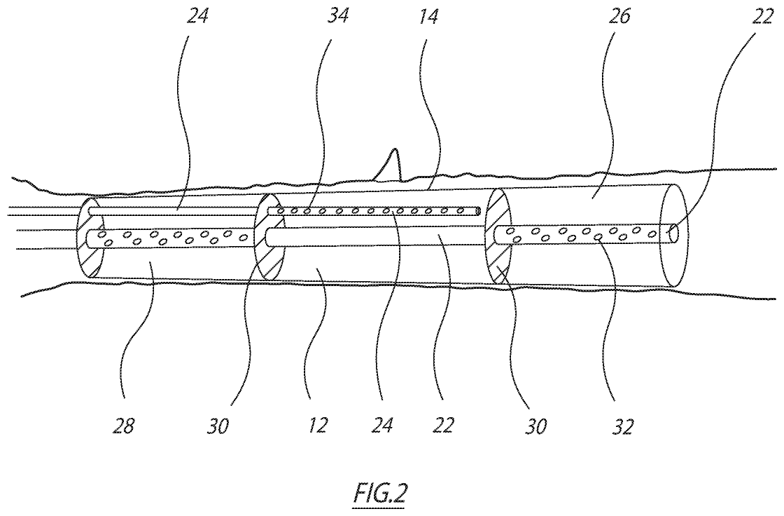

A further prior art vacuum assisted therapy device 10 is shown in FIG. 2. This device differs from that shown in FIG. 1 by the provision of a respective further foamed sponge absorbent components 26 and 28 disposed forwardly and rearwardly of the absorption element 12, the further sponges 26 and 28 being separated from the absorption element 12 by a respective juncture 30 sealing the absorption element against egress of absorbed bodily substances into the absorption element 12 from the sponges 26 and 28.

As shown in FIG. 2, the drainage tube 22 extends through both of sponge elements 26 and 28 and is provided with peripheral openings 32 for entry of bodily fluids and gases from sponges 26 and 28. The suction tube 24 also passes through sponge 26 into the absorption element 12 and is provided with peripheral openings 34 for application of the applied suction to the absorption element 12. Embodiments as illustrated in FIG. 1 and FIG. 2 are described in International Patent Applications WO 2012/071626 and WO 2013/181686, the contents of each of which are incorporated herein in their entirety by cross-reference.

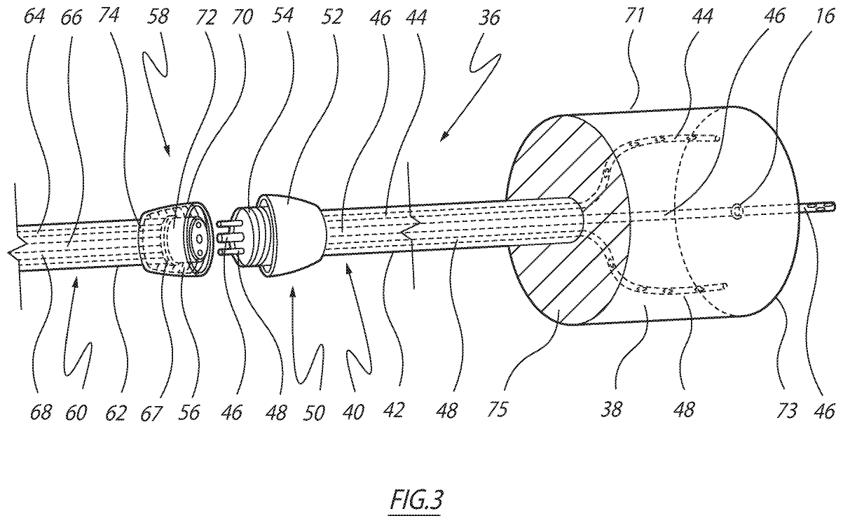

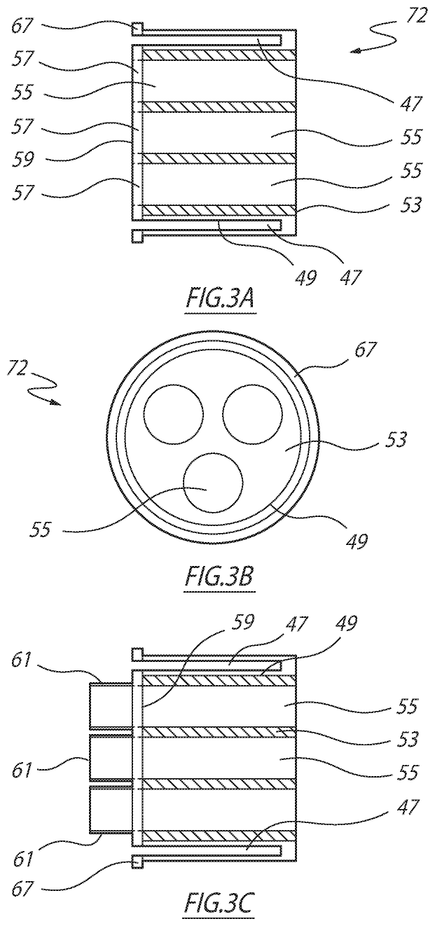

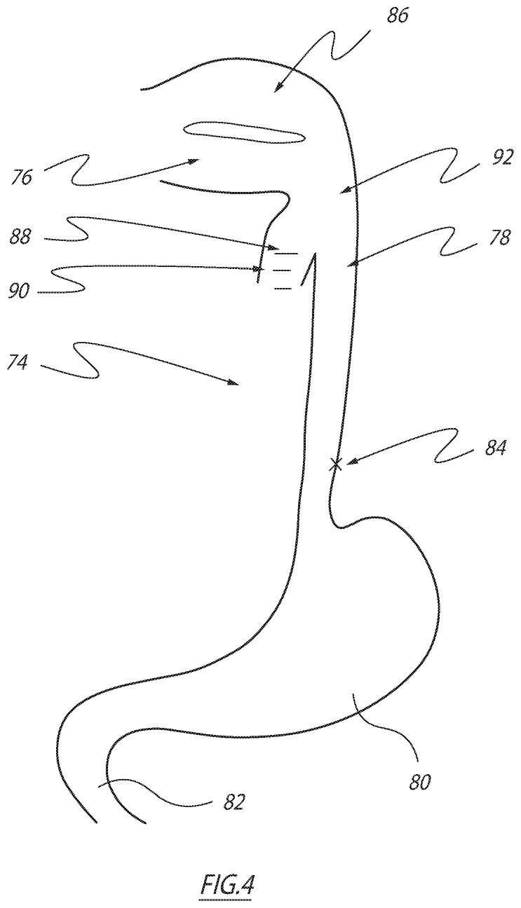

Embodiments of vacuum assisted therapy devices in accordance with the present invention are provided for placement of the device in the upper gastrointestinal tract via the mouth of the patient whilst allowing suction to be applied to the device through the nasal passage of the patient following the location of the device. A device 36 embodied by the invention is illustrated in FIG. 3.

As shown in FIG. 3, the device 36 includes an absorption element 38 in the form of a cylindrical foamed sponge connected to tubing 40. The tubing 40 comprises an outer tube 42 and a plurality of separate inner tubes 44-48, each of the inner tubes providing a respective internal lumen. The inner tubes respectively protrude from a coupling fitting 50 on a distal end of the tubing 40. The coupling fitting 50 has an outer portion 52 the diameter of which reduces in the distal to proximal direction of the fitting 50. As also shown, an engagement portion 54 of the coupling fitting is provided with a male thread for mating with a corresponding female thread 56 defined on a further coupling fitting 58 disposed on the distal end of an extraction element in the form of extraction tubing 60 used for withdrawing the distal end of tubing 40 of the device 36 through the nose of the patient in use as further described below.

The further extraction tubing 60 also comprises an outer tube 62 and inner tubes 64, 66 and 68 corresponding to inner tubes 44, 46 and 48 of the tubing of the device 36. The coupling fitting 58 of the further tubing comprises an outer portion in the form of a rotatable collar 70 the diameter of which reduces in the proximal to distal direction of that fitting and is rotatable relative to the outer tube 62 and a base portion in the form of an end cap 72 which is mounted in a fixed position on the distal end of the outer tube 62. The collar 70 has an inwardly directed flange 74 defined at its distal end.

To couple the tubing 40 of the device 36 to the further tubing 60, the protruding ends of the inner tubes 44-48 of the tubing 40 are respectively sealingly received by the end cap 72 of the further coupling fitting 58 to align with the corresponding one of the inner tubes 64-68 of the tubing 60 as described further below by way of example with reference to FIGS. 3A to 3C. As with other embodiments illustrated herein, the end caps shown in FIGS. 3A to 3C are not drawn to scale.

As illustrated in FIG. 3A, the end cap 72 in the embodiment shown is a plastic ferrule provided with an annular cavity 47 in its outer peripheral region for reception of the side wall of the outer tube 62 of the extraction tubing 60. A well 49 is defined in an opposite end of the end cap and a plug body 53 of the cap is received in the well so as to be seated on the floor of the well. A respective through passageway 55 is defined in the plug 53 for each of the inner tubes and is aligned with a corresponding through aperture 57 defined in the distal end 59 of the cap 72. As will be understood, the proximal end of each inner tube 64-68 of the extraction tubing 60 is received by the end cap through a respective one of the through apertures 57 so as to be located in the corresponding passageway 55 for alignment with the inner tubes 44-48 of the tubing 40.

When connecting the coupling fittings 50 and 58 together the distal end of each of the inner tubes 44-48 of the tubing 40 is inserted into the opposite end of the corresponding passageway 55 of the plug body in a friction fit as the coupling fittings 50 and 58 are brought together. The collar 70 of the coupling fitting 58 is then advanced and threadably mated with the coupling fitting 50. As the coupling fittings are mated together, the inwardly directed flange 74 of the coupling fitting 58 engages with an outwardly directed flange 67 provided at the distal end of the end cap 72 of that fitting, whereby the inner tubes 44-48 are driven into the end cap 72 of the coupling fitting 58 with rotation of the collar 70 to secure the coupling fittings together and form sealed connections between the respective ones of the aligned inner tubes 44-48 and 64-68. A view of the proximal end of an end cap 72 in accordance with the instant disclosure is illustrated in FIG. 3B.

In embodiments, the inner tubes 64-68 of the extraction tubing 60 can be located to extend only partway along the passageways 55 of the plug body to provide for insertion of the inner tubes 44-48 of tubing 40 into the plug body 53 and be secured to the plug body by e.g., heat or sonic welding, a suitable adhesive, or be received in the plug body in a friction fit. The outer tube 62 of the extraction tubing is likewise secured in position within the annular cavity 47 of the end cap by any suitable method (e.g., heat or sonic welding etc).

In other embodiments, the inner tubing 44-48 may be inserted the full length of the plug body whereby each inner tube 44-48 is received in the open end of the corresponding inner tube 64-68 of the extraction tubing in a friction fit when the coupling fittings are mated together. In such embodiments, the inner diameter of each of the inner tubes 64-68 is dimensioned to receive the distal end of the corresponding one of the inner tubes 44-48 of the tubing 40.

In still other embodiments, rather than the inner tubes 64-68 of the extraction tubing being inserted in the plug body 53 of the end cap 72, each of the inner tubes may be sealingly fitted to a corresponding spigot 61 projecting from the distal face of the end cap as generally illustrated in FIG. 3C. Likewise, in yet further embodiments, the end cap may be provided with respective proximally directed spigots for sealingly mating with the corresponding inner tube 44-48 of the tubing 40 rather than plug body 53, when the coupling fittings 50 and 58 are secured together.

In embodiments in which the end cap 72 of coupling 58 includes a plug body 53, the plug body can be formed from a resilient plastics material to facilitate reception of the inner tubes 44-48 and 64-68 as applicable, and thereby the formation of sealed joints between the aligned inner tubes 44-48 and 64-68 of the tubing 42 and 62. Further, in embodiments including a resilient plug body 53, the proximal end face of plug body may press against the distal end face of the engagement portion 54 of the coupling fitting 50 to seal about respective ones of the aligned inner tubes 44-48 and 64-68.

The coupling fitting 50 can be secured to the distal end of the tubing 50 in any suitable such manner whereby the inner tubes 44-48 protrude from and are fixedly held and supported by the engagement portion of that fitting, and the outer tube of tubing 50 is fixedly secured to the coupling fitting 50. As above, the outer tubing 40 may be received in an annular recess of that coupling fitting and/or be fixed to the fitting by heat or sonic welding etc). In other embodiments, respective of the inner tubes 44-48 may be sealingly fitted to a corresponding spigot provided on a proximal end of the fitting 50 which opens to a spigot on the distal end of the fitting for insertion into the corresponding passageway 55, open end of the of corresponding inner tube of the extraction tubing 60 or e.g., for sealing engagement by a corresponding spigot or connector provided on the coupling fitting 58 in use. Different combinations of the above means for fixing of the tubing 40 to the coupling 50 are also expressly encompassed. Whilst the open ends of the inner tubes of the tubing 42 and 62 are illustrated as being arranged in a linear fashion in the coupling fittings 50 and 58 in FIG. 3 and FIGS. 3A and C, this is for illustrative purposes only and the endings of the inner tubes can be arranged in the coupling fittings 50 and 58 in any suitable manner, such as exemplified in FIG. 3B. Likewise, it will be understood that any suitable alternative male and female coupling fittings and coupling fitting arrangements for the coupling of tubing 40 and 60 and for being drawn through the nasal passage whilst avoiding and/or minimising nasal trauma to the patient can be utilised.

From the above, the coupling formed by the coupling fittings 50 and 58 thereby secures the tubing 40 of the device and the further tubing 60 to each other. Further, separate conduits to the absorption element are formed by the alignment of the inner tubes 44-48 with the inner tubes 64-68 as described above. In the embodiment shown, the inner tubes 44-48 of the tubing include a suction tube 44 for applying suction from an external suction source to the peripheral face 71 of the absorption element 38, an access tube 46 which opens to forward of the absorption element and which can act as a drainage tube or a feeding tube for delivery of nutrients and/or fluids into the gastrointestinal tract of the patient as may be needed, and an irrigation tube 48 for delivery of a physiologically acceptable irrigation fluid into the absorption element 38 for maintaining the absorption element moist and/or for inhibiting clogging of the device from exudate entering the absorption element from the site in the surrounding wall of the gastrointestinal tract being treated.

Again, the proximal and distal ends 73 and 75 of the absorption element 38 are sealed against egress of bodily substances present in the gastrointestinal tract of the patient into the absorption element such as saliva, gases and other bodily substances. To seal the proximal and distal ends of the absorption element, an occlusive barrier such as a suitable, flexible sheet or film of plastics material can be affixed to the respective ends in any suitable manner such as by an appropriate adhesive, heat, sonic welding, or other method. As alternative options, a sealing coating can be applied to the proximal and distal ends or for instance, the end regions of the absorption element may be fabricated from a closed cell foamed plastics material.

Further, the interior of the absorption element 38 is sealed from the interior of the tube 46 along the entire length of that tube within the absorption element. That is, there are no through openings in the side wall of the tube 46 between the distal and proximal ends of the absorption element, ensuring the suction applied via suction tube 44 is applied to the peripheral face of the absorption element. To further prevent leakage into the absorption element 38 from the through passageway 16, the surrounding wall defining the through passageway can also be sealed by e.g., application of a sealing coating as further described below

In some embodiments in accordance with the invention, the tubing 40 may connect eccentrically to the absorption element or at a position radially spaced from the through passageway 16 whereby the through passageway is clear and unobstructed from the distal end to the opposite proximal end of the absorption element. In such embodiments the surrounding wall of the absorption element 38 defining the through passageway will also normally be sealed so as to be impermeable to egress of bodily fluids, gases and other bodily substances into the absorption element from the interior of the through passageway. For example, in such embodiments, the surrounding wall defining the through passageway 16 may be defined by a suitable closed cell foamed plastics material or again, be sealed by a suitable applied sealing coating.

Thus, bodily substances in the upper gastrointestinal tract of the patient are separated from exudate and bodily substances entering the peripheral face of the absorption element (e.g., fluids, blood, cellular material etc from the site in the endoluminal surface being treated) under the action of the suction applied via the suction tube 44 in use, thereby reducing the risk of clogging and/or fouling of the absorption element surface.

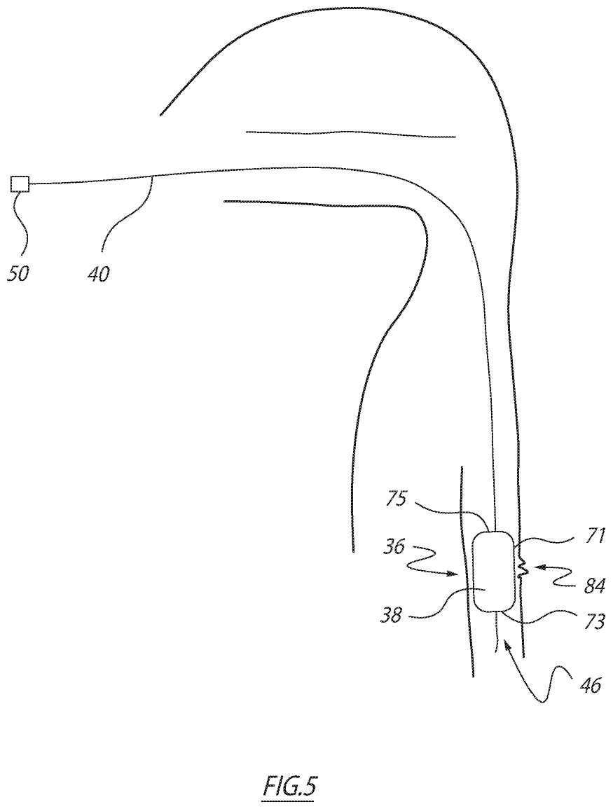

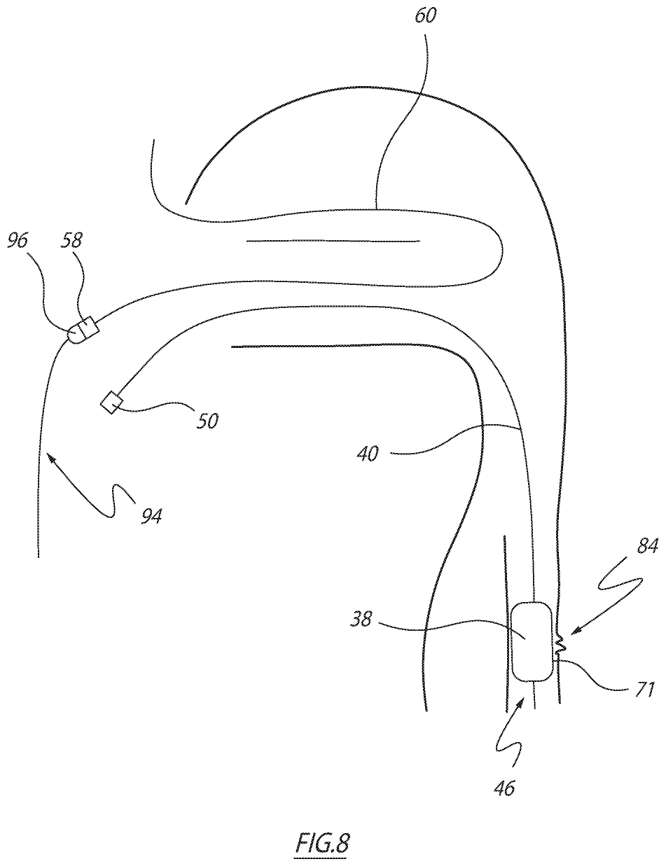

The placement of a vacuum assisted therapy device in accordance with a method embodied by the invention will now described with reference to FIGS. 4-10.

The upper gastrointestinal tract 74 of a patient is diagrammatically represented in FIG. 4. The upper gastrointestinal tract includes the mouth 76, oesophagus 78, stomach 80 and duodenum 82.

The nasal cavity of the patient is indicated by the numeral 86. The larynx and trachea are respectively indicated by the numerals 88 and 90. It will be understood that various sites below the mouth of the patient can be treated including in the duodenum.

As shown in FIG. 5, a vacuum assisted therapy device embodied by the invention is firstly inserted down the upper GI tract of a patient via the patient's mouth so that the outer peripheral face 71 of the absorption element 38 lies alongside the site (indicated by the numeral 84) in the endoluminal surface to be treated.

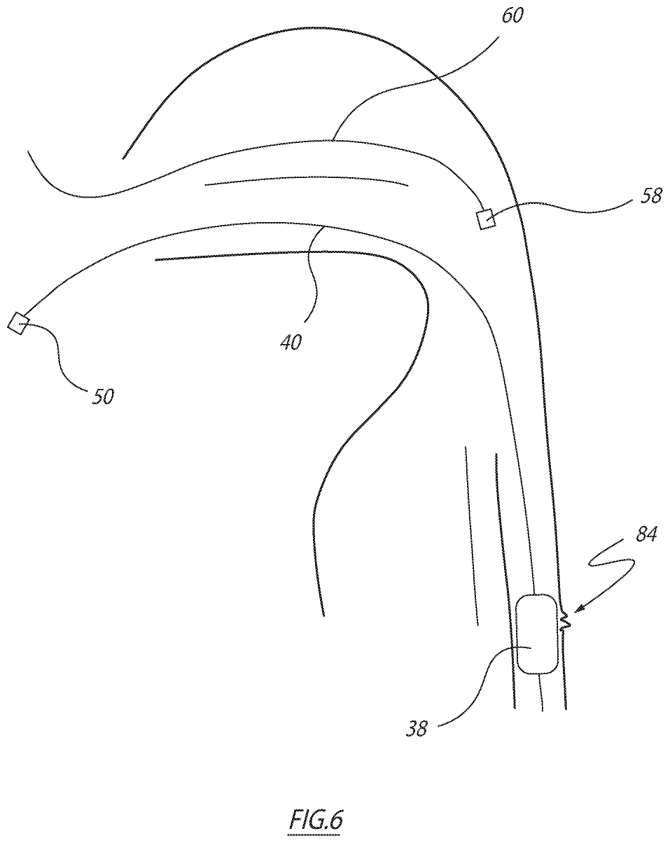

Extraction tubing in the form of further tubing 60 having a coupling fitting 58 on its proximal end is then inserted through the nasal cavity 86 to the back of the patient's throat 92 where it can be seen through the patients mouth as illustrated in FIG. 6. The coupling fitting 58 carries an exposed magnetically attracted material (e.g., a ferrous metal) not shown.

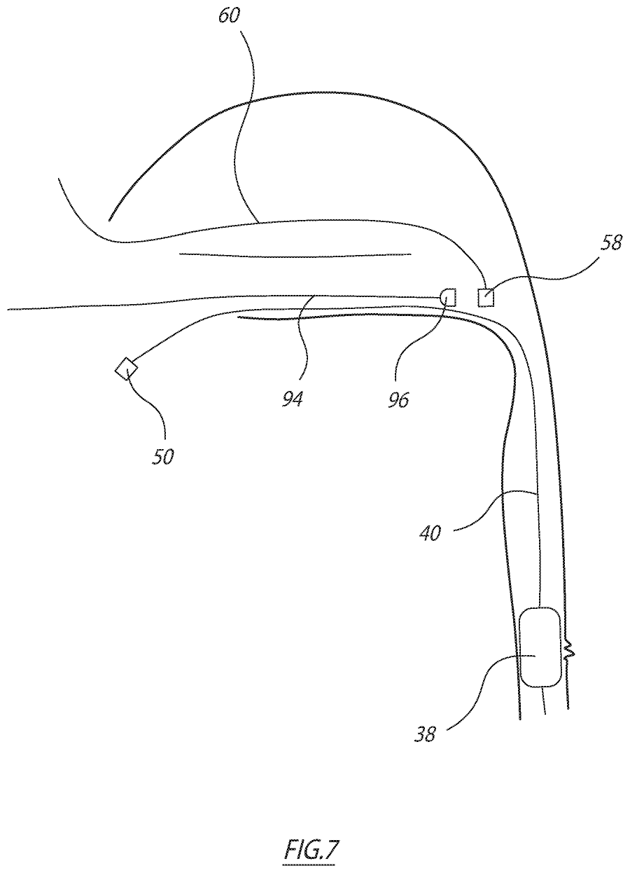

A retrieval element 94 in the form of a relatively stiff elongate probe, stylus or the like with a permanent magnet indicated by the numeral 96 mounted on its leading end is then inserted into the patient's mouth into contact with the exposed magnetically attracted material of the coupling fitting 58 whereby the magnet magnetically couples with the magnetically attracted material as illustrated in FIG. 7. The proximal end of the further tubing 60 is then withdrawn from the patient's mouth by simultaneously feeding the further tubing 60 into the nasal cavity whilst withdrawing the probe 94.

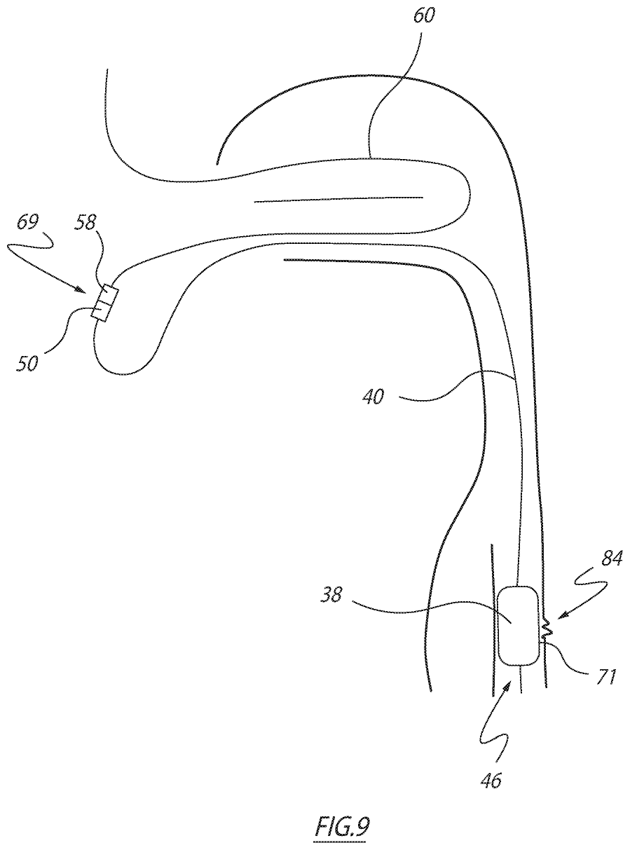

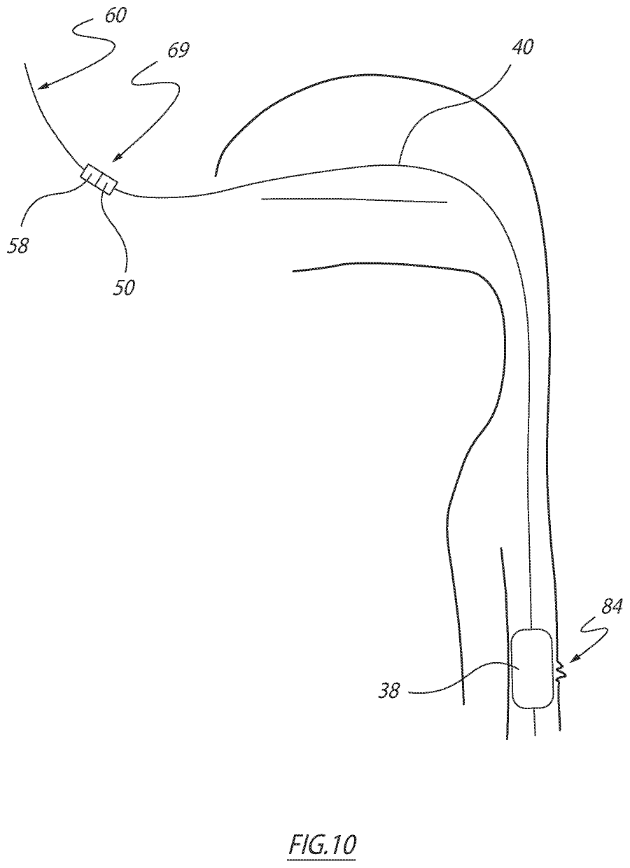

The magnetically attracted material on the proximal end of the further tubing typically comprises a protruding removeable metal end cap e.g., threadably mating with the internal female thread of the coupling fitting 58. The end cap advantageously retains the collar 70 of the coupling fitting in position. Where provided, the end cap is removed and the coupling fittings 50 and 58 are then connected together securing the tubing 40 of the device and the further tubing together, as described above and as illustrated in FIG. 9.

Once the further tubing 60 and the tubing 40 of the device 36 are securely connected together, the distal end of the tubing 60 of the device is fed back into the patient's mouth whilst simultaneously withdrawing the further tubing 60 from the nasal cavity until the coupling 69 formed by coupling fittings 50 and 58 is drawn clear of the patient's nose as illustrated in FIG. 10. The exposed tapered portion of respective of the coupling fittings facilitates passage of the coupling 69 in each direction through the nasal passage and relevant one of the nares of the patient. Desirably, the coupling fittings are smooth, soft to the touch and flush with one another and further, are dimensioned to minimise nasal trauma to the patient.

The distal end of the further tubing can terminate in an any suitable end cap fitting which provides for connection of respective of the distal ends of the inner tubes e.g., 64-68 of the further tubing 60 to a suction source, irrigation source or e.g., a conventional waste collection cannister etc, as applicable. For example, the inner tubes 64-66 may sealingly pass through the distal end cap and be provided with respective end fittings or connectors for connection to the suction source etc or allow access to the absorbent element/sponge 38 as appropriate. Alternatively, the distal end cap of the tubing 60 may have proximally directed spigots to which inner tubes 64-68 sealing mate and which open to external spigots for connection to the suction source etc or allow access to the absorption element/sponge 38. The subsequent removal of the device 36 from the patient is essentially a reversal of the above steps although for removal of the distal end of the tubing 40 of the device from the patient's mouth, the tubing 40 can be simply gripped by elongate medical tongs, forceps or the like, and withdrawn. Once the distal end of the tubing 40 is free of the patient's mouth the absorption element 38 is then carefully withdrawn from the upper G.I tract and removed via the patient's mouth.

Rather than using a ferrous metal cap as described above, the further extraction tubing 60 may for instance be provided with a ferrous metal ring on the coupling fitting of that tubing or e.g., about the tubing immediately behind the coupling fitting, for magnetically coupling with the permanent magnet of the retrieval element/probe 94. In further embodiments, the permanent magnet may be carried in the further tubing 60 and the magnetically attracted material may be provided on the retrieval element 94. In still yet other embodiments, the magnetically attracted material can be a further permanent magnet that is oppositely poled (i.e., N-S or S-N) to the permanent magnet carried on the other of the further tubing 60 or the retrieval element.

Alternatively to the above, rather than inserting the further tubing 60 through the nose of the patient to the back of his or her mouth/throat for coupling with the tubing 40 of the device 36 as described above, an extraction element in the form of an e.g., a thin flexible solid or tubular plastic rod with an appropriately profiled and dimensioned coupling fitting for being passed through the nares mounted on its proximal end for mating with the coupling fitting 50 of the tubing 40 of the device may be used instead, wherein the coupling fitting of the rod carries a magnetically attracted material or magnet for retrieval of the proximal end of the rod from the patient's mouth with use of the relevant retrieval element employed. In such embodiments, the rod can be decoupled from the tubing 40 once the distal end of tubing has been drawn from the patient's nose by the rod, and the further tubing 60 can then be coupled to the distal end of the tubing 40 protruding from the nose of the patient.

In at least some vacuum assisted therapy devices embodied by the invention, the drainage inner tube 46 when provided, can protrude from the distal face of the absorption element 38. Peripheral through openings may also be provided in the protruding proximal end region of the drainage tube to facilitate entry of bodily gases and fluids/liquids present in the upper G.I. tract into the drainage tube as illustrated in FIG. 1.

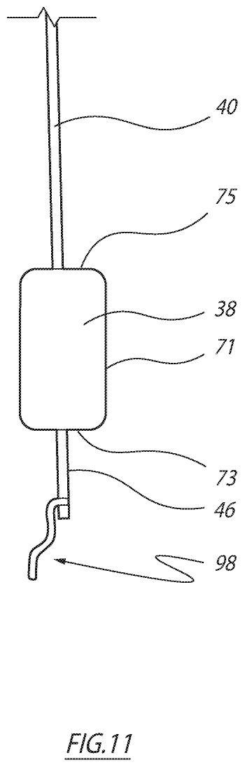

In the embodiment shown in FIG. 11, the device 36 further includes a flexible tag 98 affixed to the protruding end of the drainage tube 46 for being grasped, for example, by forceps of an endoscope for drawing the device 36 along the upper G.I tract with further insertion of endoscope whereby the outer peripheral face of the absorption element 38 is located in position immediately adjacent the site in the endoluminal surface of the G.I. tract to be treated. The tag 98 is in the form of a length of woven cotton umbilical tape wrapped at one end around the drainage inner tube 46 to which it is affixed by a suitable adhesive though any other means for attaching to tag may also be employed. However, any appropriate plastics tape or fabric strip can be utilised for the purposes of the tag, which in yet other embodiments may be provided in the form of a looped cord connected to the protruding end of the drainage inner tube 46. As will be understood, when locating the absorption element 38 of the vacuum assisted therapy device 36 in position, the leading end of the endoscope may be slid past the outer peripheral face absorption element to access the tag 98. Further, whilst in the embodiment shown the tag 98 is attached to the drainage tube 46, the tag may instead be attached to the protruding end of an e.g., an access, feeding or other inner tube of tubing 40, or any other suitable protruding support member provided for this purpose.

Embodiments of a vacuum assisted therapy device in accordance with the invention can also further comprise a respective absorbent component 100 located proximal to and/or distal to the absorption element 38 as generally illustrated in FIG. 2, wherein the juncture 30 between each absorbent component 100 and the absorption element 38 is an impermeable barrier sealing against passage of bodily fluids, gases and substances from the absorbent component(s) into the absorption element 38 as described in WO 2013/181686, the contents of which is incorporated herein in its entirety. As with the absorption element 38, each cylindrical absorbent component 100 is also dimensioned to press against the surrounding wall defining the upper G.I tract along the length the absorbent component. The absorbent component acts to soak up fluids in the G.I tract, thereby further reducing risk of fouling of the absorption element 38. Each juncture 30 can, for instance, also be provided by an occlusive adhesive coating or a flexible sheet of plastics material to which corresponding ends of the absorbent component 100 and the absorption component are affixed.

As will be understood, the inner tubing of the tubing 40 passes sealingly through each juncture 30 as required or applicable to the particular embodiment, as does the through passageway 16 when provided. Further, the tubing 40 can be connected to the distal end of an absorbent component 100 where the outer tubing 62 essentially terminates and from which the inner tubing of tubing 40 continues through that absorbent component 100 to the absorption element 38 and to a further absorbent component 100 connected to the proximal end of the absorption element 38 when provided and as applicable. In least some embodiments, a drainage, access or feeding inner tube can also protrude from the proximal end of an absorbent component 100 disposed forwardly of the absorption element 38.

In embodiments of a vacuum assisted device in accordance with the invention provided with a distal and/or proximal absorbent component 100 and having a through passageway 16 which does not receive tubing 40, the surrounding wall defining the through passageway 16 in the respective absorbent component is also sealed against entry of fluids and gases from the through passageway as described above. In some embodiments, the exposed distal and/or proximal end of the respective absorbent component 100 may also be otherwise sealed against egress of bodily substances from the upper G.I. tract.

In yet further embodiments, the absorption element 38 of a device 36 in accordance with the invention may include an expandable element for being expanded once the absorption element 38 is in position adjacent the site in the endoluminal wall of the upper G.I. tract to be treated in order to press the outer peripheral face of the absorption element 38 against the site. Typically, the expandable element is centrally located about a through passageway 16 of the absorption element. The expandable element may for instance be an inflatable inner core of the absorption element which can be inflatable via an inner tube of the tubing 40. In other embodiments, the expandable element can be provided by a resilient material biased to an expanded resting state as described in WO 2012/071626, the contents of which is also expressly incorporated herein in its entirety. In such embodiments, the expandable element may be collapsed by application of suction/negative pressure to the expandable element via the same or a different inner tube the tubing 40, and all such embodiments are expressly encompassed. Hence, the term “expandable element” as used herein encompasses elements that can be expanded from a collapsed, compressed or deflated condition in use. To provide for the expansion of the expandable element, slack may be provided in the inner tube(s) within the absorption element 38.

In the embodiment shown in FIG. 3, the tubing 40 includes a plurality of inner tubes 44-48. In other embodiments, the tubing 40 may only include a single inner tube (i.e., a suction tube for application of negative pressure to the outer peripheral face of the absorption element) whilst in still other embodiments, the tubing 40 may only include two inner tubes, at least one of which is a suction tube for applying the negative pressure to the absorption element. Thus, in embodiments described herein, the tubing 40 and further tubing 60 may only each include a single inner tube for formation of a suction tube/lumen for application of suction to the endoluminal surface via the outer peripheral face of the absorption element.

Still further, in an embodiment in accordance with the present disclosure, the coupling fittings 50 and 58 of the tubing 40 and further tubing 60 may be configured for being fitted together whereby one fitting is rotated relative to the other fitting to align the inner lumen(s) of the tubing 40 with the corresponding inner lumen(s) of the further tubing 60 to provide respective conduits to the absorption element 38 and to lock the fittings together forming a coupling as generally indicated by the numeral 69. Such arrangements may utilise a bayonet connector system in which one of the coupling fittings of the tubing 40 and further tubing 60 has one or more radially directed locking pins and the other of the coupling fittings has respective generally L-shaped slot(s) for receiving the respective corresponding pin(s). As in conventionally known bayonet cap fittings, the closed end of the short arm of the L-shaped slot is upturned for retention of the pin(s) in the slot(s) against rotation and release of the coupling fittings from one another. In such embodiments, the coupling fittings may be biased away from each other when the locking pin(s) are received in the corresponding bayonet slot(s) by e.g., the resilience of the plastics material from which the coupling fittings are made as a result of the coupling fittings being pressed together for mating of the coupling fittings, or via any other suitable arrangement. When coupled together, respective of the joint(s) formed by the alignment of the lumen(s) in the tubing 40 with the lumen(s) of the further tubing 60 are again essentially sealed against leakage. In at least some embodiments, the sealing of respective of the joint(s) may be achieved by face-to-face contact of surface(s) of the coupling fittings (e.g., the internal floor of the fittings) about the open end of each inner tube of the tubing 40 and further tubing 60. Alternatively, only a collar of one of the coupling fittings may be rotatable for location of the locking pin(s) in the bayonet slot(s) of the other fitting to form a bayonet connection whereby the inner tubes(s)/lumen(s) of the respective tubing 40 and 60 remain in stationary positions aligned with one another during the coupling of the tubing together. In this embodiment, the rotatable collar may have an inwardly directed flange for bearing against an outwardly directed flange of a base portion of that fitting for retaining the coupling fittings of the tubing 40 and 60 together, as generally shown in the arrangement illustrated in FIG. 3. Luer-lock type couplings/coupling fittings have particular application for connection of the tubing 40 to further tubing 60 in accordance with the present disclosure.

As described above, the coupling formed by the connection of the coupling fitting of the tubing of a vacuum assisted therapy device with the extraction element or coupling fitting of the further tubing in accordance with the instant disclosure are generally profiled (e.g., tapered) and dimensioned to facilitate passage of the coupling through the nasal cavity and nose of the patient as described herein. Further, in at least some embodiments, the coupling or coupling fittings (e.g., coupling fittings 50 and 58) connecting to the tubing 50 desirably have at least some degree of compressibility for facilitating passage through the nasal passage to minimise or avoid patient and/or skin tissue trauma during location of the extraction element/tubing (e.g., 60) within the nasal passage and withdrawal of the tubing 40 through the nasal passage.

From the above, the inner tubing/lumens of tubing 40 can therefore, for instance, provide respective inner tubing/lumens selected from suction lumens, drainage lumens, access lumens, feeding lumens, lumens for inflating and/or deflating an expandable element as described above, irrigation lumens and the like. In at least some embodiments, inner tube(s) of the tubing 30 defining irrigation lumen(s) can open into a drainage tube e.g., 46 at one or more locations along the drainage tube to minimise the risk of blockage of the drainage tube. An inner suction tube of the tubing 40 for applying the negative pressure to the outer peripheral face of the absorption element can likewise have spaced outlets within the absorption element e.g., in the form of peripheral openings or spigots, for distributing the application of the suction/negative pressure along the absorption element. Similarly, an irrigation tube of tubing 40 can likewise have outlets (e.g., peripherally located holes or spigots of the tubing) spaced apart along the absorption element 38 for distributing the irrigation fluid (e.g., sterile water) along the absorption element.

Whilst the provision of an inner drainage tube/lumen is desirable in an embodiment of the invention is desirable, it is not essential and embodiments of devices of the invention can be provided without one. In such embodiments, bodily substances present within the upper G.I. tract may simply pass along through the through passageway 16 of the device once the device is located in position. When provided, the drainage tube can be connected to a programmable or other electric suction pump for drawing the bodily substances through the device 36 to assist drainage and sanitary external collection of the substances from the patient for monitoring and/or disposal.

The external suction source connected to the suction tube for applying the suction to the outer peripheral face of the absorption element 38 may also be an electric pump through any suitable source of suction can be utilised. In embodiments of a device that is provided with a suction collapsible resilient insert normally biased to an expanded state as described above, the insert may be connected to the same or different suction source as the absorption element 38 to facilitate the collapse of the expandable element and thereby the insertion of the absorption element in the upper G.I tract.

Typically, the suction applied to the absorption element via the suction tube for application to the site in the surrounding endoluminal wall of the upper G.I. tract will be in the range of from 11 mm Hg to 140 mm Hg and most usually, in a range of from 50-100 mm Hg.

The absorption element 38 is typically in the form of a sponge. Various types of sponge material can be used in the fabrication of the absorption element, non-limiting examples of which include open cell polyurethane and polyvinyl alcohol foams with or without a reticulated cell structure. The pores of the sponge may be in a range of about 200 μM. to about 600 μM and generally, in a range of from about 400 μM to about 600 μM. Desirably, the foam employed is essentially non-adherent to the wound. Alternatively, the vacuum assisted therapy device in accordance with the invention can be removed before any deleterious adherence to the wall of the G.I. tract or tissue ingrowth into the sponge occurs. An absorbent component 100 as described herein can be formed from the same or different sponge material to that of the absorption element 38.

The site in the endoluminal surface of the upper G.I. tract to be treated utilising a device in accordance with the invention can for instance be a wound arising injuries due to accidents or trauma, surgery (e.g., surgical resection), disease or medical treatments such as ablation, radiotherapy and chemotherapy.

The coupling fittings and tubing 40 and further tubing 60 can be of any suitable medical grade plastics and silicone materials conventionally used in the fabrication of tubing and medical components for in vitro and/or in vivo use as appropriate, including combinations of such materials.

Embodiments of vacuum assisted therapy devices and methods in accordance with the invention may therefore provide one or more of the following:
Positioning of the absorption element 38 of the device in the upper G.I tract via the mouth of the patient with suction applied to the absorption element via the nose of the patient;
Retention of the mouth of the patient free e.g., for endoscopic placement of the absorption element adjacent the site in the G.I tract to be treated; and
Removal of the absorption element 38 from its position in the upper G.I tract via the mouth of the patient.

As will also be understood, step(s), feature(s) and/or integer(s) of an embodiment as described above may be implemented in conjunction with step(s) feature(s) and/or integer(s) of other embodiments, e.g., aspects of one embodiment may be combined with aspects of another embodiment to realize yet further embodiments within the scope of the present disclosure, and all such arrangements, alternatives and embodiments are expressly provided for herein.

Although a number of embodiments of the invention have been described above it will be understood that various modifications and changes may be made thereto without departing from the scope of the invention. The embodiments described above are therefore only illustrative and are not to be taken as being restrictive.

1. A vacuum assisted therapy device for applying a negative pressure to a site in an endoluminal surface in the upper gastrointestinal tract of a patient, the device comprising:
an absorption element for application of suction to the endoluminal surface via an outer peripheral face of the absorption element, the absorption element having a proximal end and an opposite distal end, and the outer peripheral surface extending between the proximal and distal ends of the absorption element;
tubing providing a longitudinal internal lumen for supplying the suction to the absorption element; and
a coupling fitting provided on a distal end of the tubing and configured for connection of the tubing in use to an extraction element for drawing of the distal end of the tubing through the nasal cavity of the patient to protrude from the nose of the patient following insertion of the absorption element into the upper gastrointestinal tract of the patient via the patient's mouth, the absorption element being connected to an opposite proximal end of the tubing.

2. The device according to claim 1, wherein the lumen of the tubing of the device for supplying the suction to the absorption element is one of a plurality of longitudinal internal lumens of that said tubing which lead to the absorption element.

3. The device according to claim 2, wherein the extraction element is further tubing comprising a plurality of further longitudinal internal lumens, and wherein the coupling fitting is for connecting the tubing of the device to a coupling fitting of the further tubing whereby respective of the lumens of the tubing of the device are aligned with corresponding ones of the lumens of the further tubing to form respective conduits to the absorption element.

4. The device according to claim 2 or 3, wherein the coupling fitting of the device is provided for mating with the coupling fitting of the further tubing to form a respective sealed connection between each said lumen of the tubing of the device and the corresponding said lumen of the further tubing.

5. The device according to claim 2 or 3, wherein the tubing of the device comprises an outer tube and inner tubing received within the outer tube, the lumens of the device being defined by the inner tubing.

6. The device according to claim 5, wherein the inner tubing comprises a plurality of tubes each defining a respective longitudinal said lumen of the tubing of the device.

7. The device according to claim 4, wherein the lumens of the device include one or more lumens opening to beyond a proximal end of the absorption element thereby providing passage through the absorption element.

8. The device according to claim 7, wherein each said further lumen opening to beyond the proximal end of the absorption element is respectively a lumen independently selected from the group consisting of a drainage lumen for drainage from the gastrointestinal tract to exterior of the patient, an access lumen for providing access to the gastrointestinal tract, and a lumen for passage of food and/or administered liquids from exterior of the patient through the absorption element into the gastrointestinal tract of the patient.

9. The device according to claim 4, wherein at least one further said lumen of the tubing of the device opens into the absorption element in addition to the suction lumen.

10. The device according to claim 9, wherein a plurality of said lumens of the device open into the absorption element in addition to the suction element.

11. The device according to claim 9, wherein each said lumen opening into the absorption element in addition to the suction lumen is respectively a lumen independently selected from the group consisting of a lumen for providing irrigation fluid internally into the absorption element, a further suction lumen for providing the suction to the peripheral face of the absorption element, and a lumen for passage of a fluid into the absorption element for expanding the absorption element to press the outer peripheral face of the absorption element against the endoluminal surface.

12. The device according to claim 11, wherein the absorption element includes an expandable element for being expanded to press the outer peripheral face of the absorption element against the endoluminal surface, and tubing of the device includes a said lumen for passage of a fluid to the expandable element for said expansion of the expandable element and withdrawal of the fluid from the expandable element.

13. The device according to claim 1, wherein the absorption element at least one through passageway extending from the proximal end and opening to the distal end of the absorption element, and the proximal end and distal end of the absorption element are otherwise sealed against egress of bodily fluids present within the upper gastrointestinal tract of the patient into the absorption element.

14. The device according to claim 1, further comprising at least one absorbent component for absorbing bodily fluids present within the upper gastrointestinal tract of the patient, the absorbent component being respectively located proximally or distally to the absorption element and separated from the absorption element by a juncture connecting the absorbent component to the absorption element, the juncture sealing the absorption element against egress of said bodily fluids from the absorbent component into the absorption element.

15. The device according to claim 14, comprising at least one absorbent component for absorbing bodily fluids present within the upper gastrointestinal tract of the patient, the absorbent component being respectively located proximally or distally to the absorption element and the through passageway extending through a juncture connecting the absorbent component to the absorption element, the juncture otherwise sealing the absorption element against egress of the bodily fluids from the absorbent component into the absorption element.

16. A method for locating a vacuum assisted therapy device in the upper gastrointestinal tract of a patient, comprising:
providing a vacuum assisted therapy device for application of negative pressure to a site in an endoluminal surface of the upper gastrointestinal tract, the device having an absorption element with an outer peripheral face for contact with the endoluminal surface, and tubing providing a longitudinal internal lumen for being connected to a suction source, the lumen of the tubing being in fluid communication with the absorption element to apply the negative pressure to the endoluminal surface via the outer peripheral face of the absorption element with operation of the suction source;
inserting the absorption element in position in the upper gastrointestinal tract via the mouth of the patient whereby the tubing of the device extends from the patient's mouth;
inserting an extraction element though the nose of the patient into the patient's throat, the extraction element having a proximal end and a distal end;
withdrawing the proximal end of the extraction element from the mouth of the patient leaving the distal end of the extraction element exterior of the patients nose;
connecting the proximal end of the extraction element to a distal end of the tubing of the device; and
withdrawing the extraction element from the nose of the patient whereby the distal end of the tubing of the device is drawn back into the throat of the patient and out of the patient's nose whereby the absorption element remains within the upper gastrointestinal tract of the patient.

17. The method according to claim 16, wherein the withdrawal of the extraction element from the mouth of the patient comprises inserting an elongate retrieval element into the patient's mouth whereby the retrieval element is used to grip or couple to the extraction element, and withdrawing the retrieval element to withdraw the proximal end of the extraction element from the mouth of the patient.

18-23. (canceled)

24. The method according to claim 16, further comprising, either;
(i) disconnecting the extraction element from the tubing of the device after withdrawing the extraction element from the nose of the patient, and connecting further tubing to the distal end of the tubing of the device, the further tubing having a coupling fitting on the proximal end of that tubing, and mating the coupling fitting of the further tubing with a coupling fitting of the tubing of the device to form a coupling connecting the tubing of the device to the further tubing whereby the lumen of the tubing of the device is in fluid communication with a corresponding longitudinal internal lumen of the further tubing; or
(ii) wherein the extraction element comprises further tubing provided with a coupling fitting on the proximal end of that tubing for mating with a coupling fitting provided on the distal end of the tubing of the device, and the connecting of the extraction element to the tubing of the device comprises mating the coupling fitting of the further tubing with the coupling fitting on the tubing of the device to form a coupling connecting the tubing of the device to the further tubing so the lumen of the tubing of the device is in fluid communication with a corresponding longitudinal internal lumen of the further tubing;
whereby the distal end of the tubing of the device is drawn from the patient's nose with said withdrawal of the extraction element.

25-27. (canceled)

31. A vacuum assisted therapy device for applying a negative pressure to a site in an endoluminal surface in the upper gastrointestinal tract of a patient, the device comprising:
an absorption element for application of suction to the endoluminal surface via an outer peripheral face of the absorption element for contact with the endoluminal surface, the absorption element having a proximal end and an opposite distal end and the outer peripheral surface extending between the proximal and distal ends of the absorption element;
tubing for supplying the suction to the absorption element; and
a tag for being gripped to draw the device along the upper gastrointestinal tract of the patient to locate the outer peripheral surface of the absorption element adjacent the site in the endoluminal surface to be treated, the tag being located forward of the proximal end of the absorption element.

32. (canceled)

33. (canceled)
